SEQUENCE LISTING (5 sequences)

SEQ ID NO: 1 - DNA, 137 bp, adeno-associated virus
  LTR (1)..(137)
tccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 2 - DNA, 4775 bp, Zebrafish
  promoter (1)..(4775)
attgcacgagatgtacgttatagggggaagaaacgctgagaaaaagtttattttcagtagaaaaatgttatttttcttctataaaccacttgttgaaacattttttcaatgtgtaaatgttgtgtaccatattacatcagtcactcttagttcccctctgtcaatctcattttggttacatttccatattaggaatctacctgtatcagtgcttagagcgagcagcctcaaccagtccatcccatcagaactctgagtttgttacctgttggtgtttccatatagggtgctccaggtctgatgggtggagacagtcagtaggccacttctctgtgtaatattattgtgttctttcattctcctatgctgtatacagtaaacaacagagcagatgttcagcaaattaaaaaaacgacaacatttaataaaagcaacaacaagagagaatcgtagactctcaatcactctgtctaatttccgctgtaagtcacatgacttcgccttggttgcaaagaactacaccatgtgactcatccacagattgccttataaggagattggctgaatatctcttaaaggggccctgaacaaagtgtctcagcaaacaagacacaacacaaaggttatctcatttccttcatatttttcattgacctatggctttggaagaaaatcatttttagttgctgtgtgtacatatgcaagctcccatcacaataaaacacacccatatgtcaaaacagctgctcagactaataaacgaacaagcaatcatgctttacaagtttaccacagagtttatatgctagacttatttccacagggctcctaaaattagctttgctcactttatatttgttttttgatagaagcctcagcaacttcctctcagatttgtcacattgtgctgtggctgctgtaacacttagtcgcctctctctctcttcctcttcctctttctgtttgaaacaaccttataagggcctcagtaatctgctgatgacagaaatgataggaaatcaaaaagttatcataaacacatatgcaatactttctagtaatcataaaagcacagtacattcaaaaatgacgtatttctcactatttactcatcctcaagtggttaaaaaccttaaagagtttcatttttctgttcaaaacacaggagatattttgaggaaagctaaaaacctgtagcctaaccattgacttgcatagtaggaaaaaaatagaaaccttttcagatttcctctacagctaaaaagaaactcccctccaccctaaacaccaagctgaaactcacacacctaggaaagaatgggcccggtatcaggaatgtagagcactttgtgaaataatcatgaggcaataaactgtacctatgactcattgacctctgtgatgaagtttatcttttctataaggctcagtgagttataggtatgacatttggattctataggtagtcaaaattcaatatttacatcactgcacgcctaggatgtgggggtgggcagatgtattgggtgtcactgctgttggaagcatcattacagacattttctgatgacaagctgctttcgtcactagataaatcacacaacagactgtttatcaagtggtgacatagccctactgacatttcaataaccatagctcctaaagattttaacattaaatgcacaatttagtcctctttttggtttcatgctgacaagataagacaagaattgcaaagccccctggataaggcatgctgccacgcgtctcagattgcccatgctaaaggcaggacattataactggcattaacatctatttatgaccccatggcctcctaaaaaacccgaataggtaaaggatttacatatttcaatgtaaattcatatcaaatcgatctgggaccttgtttagccaaaataaacgctgtgtagtattttggtttctttcatgatagcatttgttgctaatagtccctacacatttatcaatgtaaactagtctttgcttcaaaacatcagcatttaacatttaagaatgggcagattagggagtgggcagatgtattaggtgtcatcctggtgacgttttaacagccattgctccttacgattttaacattaaatgcacaatttagtcccctttttggtttcatgctgataagataagaattgcaaagcaccatggatagggcgtgctgccacgcgtctcagattgcccaagctgaaggcaggacatttaaaactgggattaacacctatttatgaccccttggttcctctgaaaaaccggaatatgctaaagaatttacatattttcaatgaaaagtcatattaaatcaatttgggatctcgtttagcctaaataggaaccctttctagtatttttttttccttgaagccattagcctagcctatcagtgtcacattactttatcgaatgtaaactaaacagtctgtgcttcaaaacattcacgacatatagtgttattcaatagtttatattatcagtctttttaagtaacagggtatgtaggctgattttttttgtgcactcttcagattccagtaatgcagcagcgtgaagcgtcatcggtttatgacgtaattacgcaaaagccggcttgacaggaagtgttgttttctatggagcaccgtgcccttgttttgtatcccctgctttgcagataaccagataaaatgaaatatgcttaatacggttgcatctaacatgccgagcggcgatgtaacagacgccgatctgagcaacaacaaaacaatagtcaatatttcattaatttccgaatgactcctgatcttctgcagctttacatccatgatattataaacagcaaagcattatgcacaaaagcaacctacgattgaattaaacagtgcaatgttcacaaataaagccttttttaaatatgcttttacattatgttttatagagtcacgagaagtacctgtactttaaatgggagccacgcccacgttgttcatcaaactaacctaataaaatgacacacttaaaatgtcttctcaagaattaatcgtaaatgagtcgttttcaactagctgtcaaacattaactgaaatgtaaagcagattagtgtataacgttaatcaaataatgatttcttctctgttctaccaataatggctcttgtataacgttagtttacacattgttgtcatttatagagctgaaacatacagtaaaatacaatataagtttaaatactattggacaatgtcgtactgctatgtttaaacgtcctgtcattcctgcagcctaagtacagcttatataaatgcaactttacatcatgcattgctcgtttttcacagaaataataaactatttaacgttactcgctgtatgtgaggttatttaaaagtaacgttaagccacagacatgaagtctagcatcctttactgatttaactgtgttccaatccaaatgatatacatatccttaattcaagtgttacatctgccaatgctgtgtcaaatcaacttatcgtgtatttccttatcaaattatttcgccttttcgccgatccctgacatacattatttcaacccagaacttgaatttcaagcaactagcttgaaactcgccaagtggacgacggctggccaatcagaccgctcgctgccgaaagtttaccttatatggaaggagccggccgcagtgccggctataaagctgtgacccacctcacgctcagcattgtgagttttcagtgcacgctgagaagatcttcactccccttgttcacaataacctactaatacacaggtgagaatgcttttatattaatatttactggtatttggaataatttagtaatgcttatgatgattattggagatttatttattaaagctatatttcattttgcttgctgatcggatttgaattgaagtcttggtcctgtctgcctaaatatgtactttgctgttgatttttacaacgctgaataagttatgctgatttttgtgcttgtgttttattaatggttttaaccggtagcagttccagctttcaaagctaacgttagagtaatacaactgtaaagccttctacataaagcgatttattaaattcatatttggtagtaaattgatttttaatgtaaggtttaatttaaatgtggatttattaatcaacgttaacgttagtatcatcaacggcagtgttcgcagtttgaatggtgggtgtggctactataacgcattaaccggtatcactgccgtttaattcgtgtgcacggtcaaatatatataaaagcttttaaagtttcttgtttgtctgtaacgttgttgggatttcataaacttgtcagtcacacacgaagttaaagattattgcagcacttctagttcgttaaagttaagttactctttctgttgaaatggcttcgttttatttccttttcaaaagacaagagcgtgttttagtgagcaccgggatttcactcgtggcgttgtaaagcgcttatctcggcctttgagaccgggagggagtggtcgcgtccgttggtctgattccacgcgctgaatcggcggcattgagctgctctaacttattgccatataaggcaatagtgaggctgcctggccacttcctttgtctcaaatctcttggtttcaaccctgcgcctttgcgccgcctactggccagacgtcacaaatccatcagtcatcaccgcctataaaagacactacccattcatattttaacagtttcgctttttttccctacagcc

SEQ ID NO: 3 - DNA, 678 bp, Discosoma coral
  Fluorescent gene (1)..(678)
atggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtag

SEQ ID NO: 4 - DNA, 53 bp, Simian virus 40
  polyA_signal (1)..(53)
gcagtgaaaaaaatgctttatttgtgaaatttgtgatgctattgctttatttg

SEQ ID NO: 5 - DNA, 137 bp, adeno-associated virus
  LTR (1)..(137)
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagaggga

(19) United States
(12) Patent Application Publication
(10) Pub. No.: US 2009/0133138 A1
(43) Pub. Date: May 21, 2009

(54) NEW GENE FRAGMENT, NOVEL TRANSGENIC ZEBRAFISH AND METHODS FOR PRODUCING TRANSGENIC ZEBRAFISH

(75) Inventor: Huai-Jen Tsai, Taipei (TW)

Correspondence Address: WPAT, PC;INTELLECTUAL PROPERTY ATTORNEYS, 2030 MAIN STREET, SUITE 1300, IRVINE, CA, 92614, US

(73) Assignees: Huai-Jen Tsai, Taipei (TW); Taikong Corp., Taipei (TW)

(21) Appl. No.: 11/940,998

(22) Filed: Nov. 15, 2007

(51) Int. Cl. A01K 67/027; C07H 21/04; C12N 15/00

(52) U.S. Cl. 800/20; 536/23.1; 435/320.1; 800/25

(57) ABSTRACT

The present invention provides a method for producing systemic red fluorescent zebrafish. The present invention also provides a new gene fragment and a systemic red fluorescent zebrafish.

### FIELD OF THE INVENTION

The present invention relates to a method for producing novel transgenic zebrafish.

The present invention also relates to a new gene fragment and novel transgenic zebrafish.

### BACKGROUND OF THE INVENTION

Transgenic ornamental fish is one sector of the fishery business and belong to entertainment industry with global business. For example, transgenic fish expressing green fluorescence by introduction of a GFP gene fused with a fish-specific gene promoter into fertilized eggs, has been generated using zebrafish (Hamada, K. et al., Mol. Marine Biol. Biotech., 1998. 7, 173-180).

Hsiao et al. disclosed a DNA construct flanked at both ends by inverted terminal repeats (ITRs) to increase the efficient expression of transgenic genes in zebrafish. A uniform transgene expression was achieved in the F0 and the following two generations (Hsiao et al., Developmental Dynamics 2001. 220: 323-336). US 2004/0117866 A1 also disclosed a similar gene fragment for producing red fluorescent zebrafish by α-actin promoter.

Although the transgenic green and red fluorescence zebrafishs have been described, method and condition of generating other transgenic fish with other gene fragment (such as red fluorescent protein expressed by β-actin promoter) is different and cannot be easily deduced from the prior art because of the different strategies of genetic construction, gene expression, gene inheritance and uncertainties of the transgenic technique.

U.S. Ser. No. 10/752,687 constructed the pβ-DsRed2-1-ITR gene fragment for producing transgenic medaka (β-actin form medaka). U.S. Ser. No. 11/235,539 used similar gene fragment for producing transgenic cichlid (β-actin form cichlid). However, the expression of the transgene may be influenced by the copy number of the transgenes, and the interactions between the transgene and its flanking genomic DNA as noted on Fraser et al. (Fraser et al. Current Opinion in Cell Biology 1998. 10:361-365). Fraser et al. reported that the site of transgene integration in the host genome will affect the transgene expression, also called the position effect.

The gene targeting is well established in the mouse; however, gene-targeting protocols have not been developed in the rat despite the establishment more than 16 years ago of the first transgenic rats by pronuclear injection (F Kent Hamra et al. PNAS 2002. 99:931-936). Therefore, the results of similar gene fragment expressed in different species are unpredictable and worth studying.

The individual promoters have different abilities to express report gene expression in ES cell and other cell types. (Chung et al. STEM CELLS 2002. 20:139-145) Thus, the same gene driven by promoters from different species is unpredictable of its expression.

### SUMMARY OF THE INVENTION

The present invention provides a gene fragment comprising (1) a β-actin gene promoter of zebrafish; (2) a gene encodes red fluorescent protein; (3) SV 40 poly-A signal; and (4) inverted terminal repeats (ITR) of adeno-associated virus.

The present invention also provides a method of producing zebrafish with systemic red fluorescence comprising:
(a) constructing a plasmid including ITR, cytomegalovirus (CMV) promotor, a fluorescent gene, S40 poly A and ITR form upstream to downstream;
(b) replacing the CMV promotor with an β-actin gene promoter of zebrafish to produce a new plasmid construct;
(c) linearizing the new plasmid construct;
(d) microinjecting the appropriate amount of linearized plasmid construct into fertilized eggs of zebrafish;
(e) selecting the eggs with fluorescence; and
(f) hatching the selected eggs to produce zebrafish with systemic red fluorescence.

The present invention further provides a zebrafish with systemic red fluorescence produced from the set forth method.

### BRIEF DESCRIPTION OF THE DRAWINGS

FIG. 1 illustrates the construction of plasmid pZβ-DsRed2-1-ITR.

FIG. 2 illustrates the inheritance/expression rates of the novel zebrafish (with transgene pZβ-DsRed2-1-ITR) of different generations.

FIG. 3 is a photographic representation of a three-month-old transgenic zebrafish from F2 generation that were derived from founders that are successfully transfected with the nucleic acid fragment of the invention, pZβ-DsRed2-1-ITR, demonstrating its red fluorescence expression.

FIG. 4 is other transgenic zebrafish expressed red fluorescence.

### DETAIL DESCRIPTION OF THE INVENTION

The current invention is of thorough and careful design with conceptual breakthrough. A plasmid construct, pZβ-DsRed2-1-ITR, could be generated by introducing the β-actin gene promoter of zebrafish into expression vector pDsRed2-1-ITR (Clontech). The appropriate amount of pZβ-DsRed2-1-ITR is then micro-injected into the cytoplasm of fertilized eggs of zebrafish prior to the first cleavage. These eggs are screened to find progeny expressing fluorescence throughout their systemic tissue. Progeny with fluorescent transgene are then used for future breeding. The term “zebrafish” in the invention is not limited but to that from D. acrostomus, D. aequipinnatus, D. malabaricus, D. albolineatus, D. annandalei, D. apogon, D. apopyris, D. assamensis, D. choprae, D. chrysotaeniatus, D. dangila, D. devario, D. fangfangae, D. frankei, D. fraseri, D. gibber, D. interruptus, D. kakhienensis, D. kyathit, D. laoensis, D. leptos, D. maetaengensis, D. malabaricus, D. naganensis, D. neilgherriensis, D. nigrofasciatus, D. pathirana, D. regina, D. rerio, D. roseus, D. salmonata, D. shanensis, D. spinosus, Brachydanio frankei, Brachydanio rerio albino and Branchydanio sp.

The gene fragment used in the present invention comprising (1) a β-actin gene promoter of zebrafish; (2) a fluorescence gene; (3) inverted terminal repeats (ITR) of adeno-associated virus; and (4) a basic part from pUC.

The red fluorescent gene can be purchased from BD Bioscience Clontech or Evrogen IP (Russia). The red fluorescent gene is DsRed2-1, DsRed2, DsRed2-N1, DsRed2-C1, TagRFP, pTurbo FP635N or pTurboFP635-C. In the embodiment of the invention, pDsRed2-1 is used as the source of the red fluorescent gene. pDsRed2-1 encodes DsRed2, a DsRed variant engineered for faster maturation and lower non-specific aggregation. DsRed2 contains a series of silent base-pair changes that correspond to human codon-usage preferences for high expression in mammalian cells. In mammalian cell cultures when DsRed2 is expressed constitutively, red-emitting cells can be detected by fluorescence microscopy within 24 hours of transfection. Large insoluble aggregates of protein, often observed in bacterial and mammalian cell systems expressing DsRed1, are dramatically reduced in cells expressing DsRed2. The faster-maturing, more soluble red fluorescent protein is also well tolerated by host cells; mammalian cell cultures transfected with DsRed2 show no obvious signs of reduced viability-in those cell lines tested, cells expressing DsRed2 display the same morphology (e.g., adherence, light-refraction) and growth characteristics as non-transfected controls. pDsRed2-1 is a promoterless DsRed2 vector that can be used to monitor transcription from different promoters and promoter/enhancer combinations inserted into the multiple cloning site (MCS).

The fragment of Claim 1, wherein the β-actin gene promoter of zebrafish is SEQ ID NO.:2.

The fragment of Claim 1, wherein the gene encodes red fluorescent protein is SEQ ID NO.:3

A plasmid comprising the gene fragment of Claim 1.

The method of the invention provides five improvements over other methods currently available:
1. The main body of the nucleic acid fragment of the invention is plasmid constructs such as pZβ-DsRed-ITR, which are commercially available at an accessible price
2. The nucleic acid fragment of the invention enables the zebrafish to emit fluorescence throughout its systemic tissue.
3. The method of the invention, which comprises microinjecting the transgene construct into fertilized eggs, ensures the transgenic zebrafish emits fluorescence at its systemic skeletal muscle at a higher ratio with better quality.
4. The heterologous transgenic fish stably passes the transgene to the next generation. Thus natural breeding could be used to maintain the transgenic population and reduces the breeding cost.
5. The fluorescence of the transgenic zebrafish, which is emitted at its systemic tissue, can be easily seen by naked eyes. The red fluorescence is further intensified under light source of shorter wavelength, providing a higher entertainment value to ornamental fish.

The present invention provides a method of producing transgenic zebrafish with systemic fluorescence comprising:
(a) constructing a plasmid including ITR, CMV promotor, fluorescent gene, S40 poly A and ITR from upstream to downstream;
(b) replacing the CMV promotor with the β-actin gene promoter of zebrafish, which directs systemic skeletal muscle β-actin gene expression, of zebrafish to produce a new plasmid construct;
(c) linearizing the new plasmid construct;
(d) microinjecting the appropriate amount of linearized construct into fertilized eggs of zebrafish;
(e) selecting eggs that shows fluorescence; and
(f) hatching the selected eggs to produce zebrafish with systemic fluorescence.

Accordingly, the preferred linearized construct is selected from

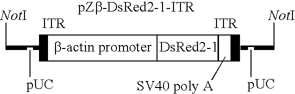

The preferred fluorescent gene used in the method of the invention is red fluorescent gene from pDsRed2-1.

In the method of producing transgenic zebrafish of the invention, the appropriate amount of linearized plasmid construct injected into the fertilized eggs is sufficient to introduce transgene into germ cell of zebrafish. The preferred amount of linearized plasmid construct injected into the fertilized eggs is 1-10 nl. The most preferred amount of linearized plasmid construct injected into the fertilized eggs is 2-3 nl.

The present invention also provides the transgenic zebrafish with systemic fluorescence produced from the method of the invention. The preferred zebrafish has systemic red fluorescence.

### EXAMPLE

The examples below are non-limiting and are merely representative of various aspects and features of the present invention.

### Example 1

### Generation of the Plasmid pZβ-DsRed2-1-ITR

Commercially available plasmid construct, pDsRed2-1 (Clontech) was used to generate the expression vector.

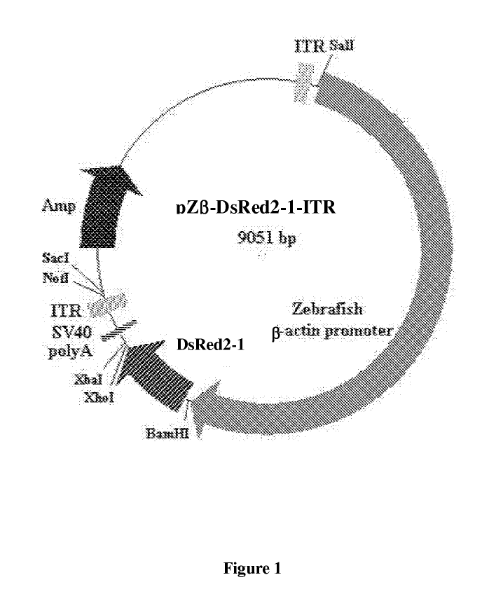

The DsRed 2-1 fragment was from plasmid pDsRed2-1. The CMV promoter and two adeno-associated virus inverted terminal repeats (ITR) were ligated to the DsRed2-1 fragment as depicted in FIG. 1 to produce plasmid construct pDsRed2-1-ITR. The plasmid construct pDsRed2-1-ITR has shown higher expression stability.

Generating the Novel Plasmid Construct: pZβ-DsRed2-1-ITR

As illustrated in FIG. 1, the zebrafish β-actin gene promoter was obtained by digesting plasmid construct pOBA-109 with restriction enzymes BamHI and SalI. BamHI was used first, ends were filled in, and a subsequent digestion with SalI provided a 4775 bp fragment.

As illustrated in FIG. 1, the CMV promoter was cut out by digesting the construct pDsRed2-1-ITR with restriction enzymes BamHI and SalI. Digestion with BamHI and SalI provided a 4240 bp fragment. Then, the β-actin gene promoter of zebrafish was inserted into the plasmid construct, pDsRed2-1-ITR, at the position where the CMV promoter was cut out. The resulting plasmid construct had two 137 bp adeno-associated virus inverted terminal repeats (ITR). One ITR (SEQ ID NO.: 5) was located at the 3′ end of SV40 poly A (SEQ ID NO.: 4). The other was located at the 5′ end of the β-actin gene promoter (SEQ ID NO.:1).

As illustrated in FIG. 1, the resulting plasmid construct, pZβ-DsRed2-1-ITR, had a total length of 9051 bps. pZβ-DsRed2-1-ITR contained (1) the zebrafish β-actin gene promoter (for systemic gene expression); (2) sea coral red fluorescent protein; (3) adeno-associated virus inverted terminal repeats; (4) SV40 poly-A signal; and (5) pUC plasmid construct basis.

Appropriate amount of pZβ-DsRed2-1-ITR was digested with proportional amount of Not I restriction enzyme. A small fraction of the digested product was analyzed by agarose gel electrophoresis to verify its linearity. The fragment length was 9051 bps as expected.

### Example 2

### Preparation of Microinjected DNA

All DNA plasmids were prepared via ultra-centrifugation with cesium chloride and ethidium bromide gradient (Radloff et al., 1967 Proc Natl Acad Sci USA 57:1514-1521). All DNA fragments used for microinjection were eluted from agarose gel following electrophoresis.

### Example 3

### Cytoplasmic Microinjection

Fish were maintained under artificial conditions of 14 h light and 10 h darkness at 26° C. and maintained on a diet of Tetramin (Tetra, Germany). Before the incubator entered the dark cycle, fish were collected and separated by separation net. On the next morning after the light cycle has begun, fish eggs were collected every 15-20 minutes.

Eggs were collected within 30 minutes of fertilization and attaching filaments removed. The linearized construct was quantified and dissolved in 5×PBS with phenol red at the desired concentration. DNA was picked up by micro-capillary of zebrafish microinjector (Drummond) wherein the injection needle width of the micro-capillary was approximately 10 μm. As micro-needle enters the cell cytoplasm, the DNA injected was approximated 2-3 nl. In each microinjection session, 30-40 eggs were injected; 250-300 eggs were injected in each experiment. Injected eggs were incubated at 26° C. in distilled water.

### Example 4

### Hatching and Screening for Transgenic Embryos

Injected eggs were rinsed with sterilized solution, cultured in incubator wherein the temperature was 28.5° C. The fluorescence could be observed in the developing embryo after 24 hours.

Embryos were observed under a bright field with a dissecting stereomicroscope (MZAPO, Leica, Germany). Dark field illumination for detecting green fluorescence was performed with a stereomicroscope equipped with a GFP Plus filter (480 nm). The distribution and intensity of the red fluorescence is observed under fluorescence microscope (Leica MZ-12; Fluorescence System: light source Hg 100 W; main emission wavelength 558 nm, and main absorption wavelength 583 nm, filter set RFP-Plus; photography system MPS60). Photographs were taken using an MPS60 camera loaded with ISO 400 film and equipped with a controller for film exposure time (Leica, Germany). In order to examine the distribution of RFP expression in the tissues of transgenic fish, 11 days of post-fertilization larva which having RFP expression on appearance were sectioned and observed under fluorescent microscopy. Larva were fixed for 30 min in 4% paraformaldehyde at 4° C., embedded in cryomatrix (Shandon, USA) and frozen at −20° C. Cryostat sections (Cryostat Microtome, HM500 OM, Microm, Germany) with 15 μm thickness were mounted on slides and observed the RFP fluorescence immediately.

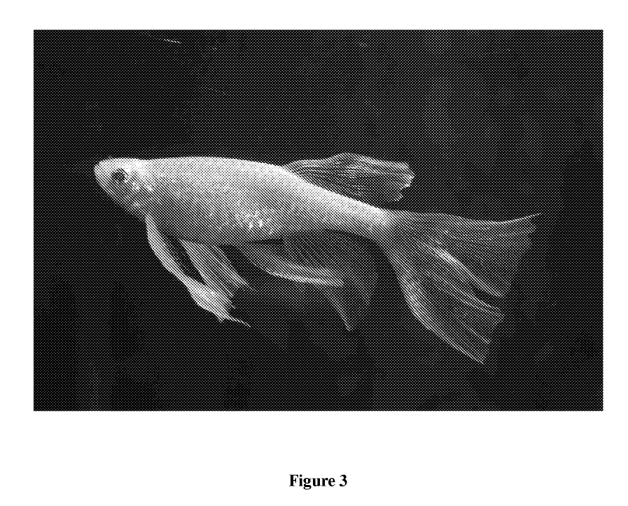

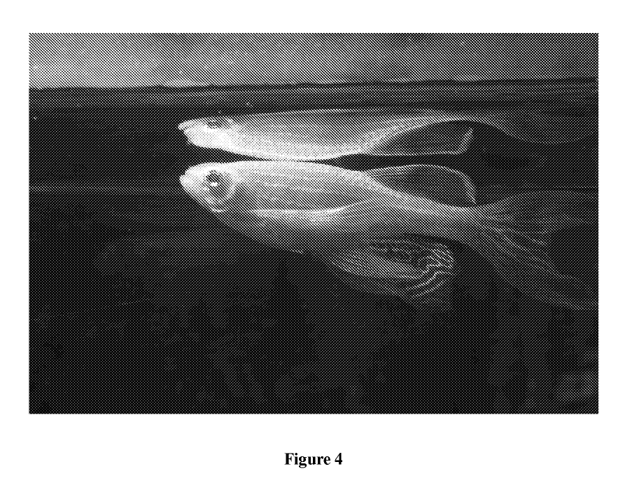

The red fluorescence fish generated from expression vector pp-DsRed2-1-ITR are shown in FIGS. 3 and 4.

### Example 5

### Germ-Line Transmission of Transgene

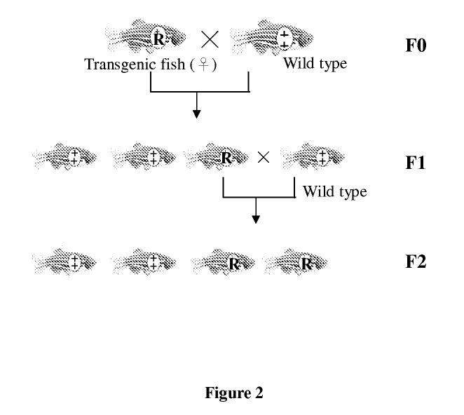

As shown in FIG. 2, red fluorescent zebrafish originated form embryos microinjected with pZβ-DsRed2-1-ITR fragment were mated with wild type, to get the progeny that exhibited uniform fluorescence. The F1 with fluorescence expression was again mated with wild type to obtain the F2 progeny (shown in FIG. 3), which all exhibited red fluorescent expression, and could be readily distinguished from fish without fluorescence expression. The difference between transgenic zebrafish and wild type could be better discerned under blue light.

While the invention has been described and exemplified in sufficient detail for those skilled in this art to produce and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. The cell lines, embryos, animals, and processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the Claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations, which are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention Claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended Claims.

Other embodiments are set forth within the following Claims.

1. A gene fragment comprising (1) a β-actin gene promoter of zebrafish; (2) a gene encodes red fluorescent protein; (3) SV 40 poly-A signal; and (4) inverted terminal repeats (ITR) of adeno-associated virus.

2. The fragment of claim 1, wherein the β-actin gene promoter of zebrafish is SEQ ID NO.: 2.

3. The fragment of claim 1, wherein the gene encodes red fluorescent protein is SEQ ID NO.: 3.

4. A plasmid comprising the gene fragment of claim 1.

5. A method of producing zebrafish with systemic red fluorescence comprising:
(a) constructing a plasmid including ITR, CMV promotor, a gene encodes fluorescent protein, S40 poly A and ITR from upstream to downstream;
(b) replacing the CMV promotor with an β-actin gene promoter of zebrafish to produce a new plasmid construct;
(c) linearizing the new plasmid construct;
(d) microinjecting the appropriate amount of linearized plasmid construct into fertilized eggs of zebrafish;
(e) selecting the eggs with fluorescence; and
(f) hatching the selected eggs to produce zebrafish with systemic red fluorescence.

6. The method of claim 1, wherein the red fluorescent gene is DsRed 2-1.

7. The method of claim 1, wherein the appropriate amount of linearized plasmid construct injected into the fertilized eggs is sufficient to introduce transgene into germ cell of zebrafish.

8. The method of claim 3, wherein the appropriate amount of linearized plasmid construct injected into the fertilized eggs is 2-3 nl.

9. A zebrafish with systemic red fluorescence produced from the method of claim 1.

10. The zebrafish of claim 5, wherein the zebrafish is from Cyprinidae.

11. The zebrafish of claim 6, wherein the zebrafish is D. acrostomus, D. aequipinnatus, D. malabaricus, D. albolineatus, D. annandalei, D. apogon, D. apopyris, D. assamensis, D. choprae, D. chrysotaeniatus, D. dangila, D. devario, D. fangfangae, D. frankei, D. fraseri, D. gibber, D. interruptus, D. kakhienensis, D. kyathit, D. laoensis, D. leptos, D. maetaengensis, D. malabaricus, D. naganensis, D. neilgherriensis, D. nigrofasciatus, D. pathirana, D. regina, D. rerio, D. roseus, D. salmonata, D. shanensis, D. spinosus, Brachydanio frankei, Brachydanio rerio albino, and Branchydanio sp.
